(19) United States
(12) Patent Application Publication
(10) Pub. No.: US 2026/0306808 A1
(43) Pub. Date: Oct. 8, 2026

(54) INTEGRATED EXTERNAL VENTRICULAR DRAINAGE AND INTRACRANIAL ELECTROENCEPHALOGRAPHY MONITORING DEVICE

(71) Applicant: The Regents of the University of California, Oakland, CA (US)

(72) Inventors: Arjun Khanna, La Jolla, CA (US); Jeffrey Tomlin, La Jolla, CA (US); Hyeonseok Kim, La Jolla, CA (US); Shadi Dayeh, La Jolla, CA (US)

(21) Appl. No.: 19/461,322

(22) Filed: Jan. 27, 2026

Related U.S. Application Data
(60) Provisional application No. 63/750,207, filed on Jan. 27, 2025.

(51) Int. Cl. A61M 27/00; A61B 5/293; A61B 5/37

(52) U.S. Cl. CPC A61M 27/006; A61B 5/293; A61B 5/37

(57) ABSTRACT

A device for bodily fluid drainage and bioelectrical monitoring is disclosed. An exemplary device includes a flexible catheter that includes perforations on the distal portion to allow bodily fluid to pass through the catheter and also includes recording electrodes on the outer surface of the catheter to contact tissue and record bioelectric signals. The device can include a trocar designed to attach to the proximal end of the catheter and secure the electrical connection to allow navigation under the skin of the patient to allow the catheter and electrical connection to be accessed away from the insertion point of the distal portion of the catheter. The device can incorporate intracranial electroencephalography (iEEG) components into an external ventricular drain (EVD) device to allow simultaneous drainage and iEEG monitoring without requiring a separate iEEG procedure.

### CROSS-REFERENCES TO RELATED APPLICATIONS

This application relates to the Applicant's patent application titled “INTEGRATED EXTERNAL VENTRICULAR DRAINAGE AND INTRACRANIAL ELECTROENCAPHALOGRAPHY MONITORING DEVICE,” U.S. Provisional Patent Application No. 63/750,207, which was filed on Jan. 27, 2025, the contents of which are hereby incorporated by reference in their entirety.

### TECHNICAL FIELD

This document generally relates to methods, devices, and systems relating to surgical drainage and monitoring, and, more particularly, to brain fluid drainage and brain monitoring.

### BACKGROUND

Intracranial electroencephalography (iEEG) is a technique used to record electrical activity from the brain using electrodes placed within or on the surface of brain tissue. iEEG device insertion involves creating a scalp incision, perforating the skull, and passing an electrode through brain tissue to a target location. iEEG can provide information about neural activity that is not detectable using scalp-based electroencephalography due to signal attenuation caused by the skull and intervening tissue layers. However, iEEG devices are underutilized in large part due to procedural morbidity associated with insertion of the device.

### BRIEF DESCRIPTION OF FIGURES

Non-limiting and non-exhaustive examples are described with reference to the following figures.

FIG. 1 shows a schematic of an example device, according to some embodiments of the present technology.

FIG. 2 shows another schematic view of the example device previously described with respect to FIG. 1, according to some embodiments of the present technology.

FIG. 3 shows a schematic of another example embodiment that can be implemented with the device of FIG. 1, according to some embodiments of the present technology.

FIG. 4 shows a schematic of an elastomer tube containing distal perforations and bonded with a thin film substrate containing embedded recording contacts and associated circuitry, according to some embodiments of the present technology.

FIG. 5 shows a portion of the example embodiment depicted in FIG. 4, according to some embodiments of the present technology.

FIG. 6 shows an example insertion of a device according to embodiments of the present technology.

FIG. 7 shows an example of intracranial components of the device, according to some embodiments of the present technology.

FIG. 8 shows a schematic of an example device, according to some embodiments of the present technology.

FIG. 9 shows another schematic view of the example device previously described with respect to FIG. 8, according to some embodiments of the present technology.

FIG. 10 shows a schematic of another example embodiment that can be implemented with the device of FIG. 8, according to some embodiments of the present technology.

FIG. 11 shows a schematic of an elastomer tube, with distal perforations, that is produced with a slight relief at the site of recording electrodes, according to some embodiments of the present technology.

FIG. 12 shows a portion of the example embodiment depicted in FIG. 11, according to some embodiments of the present technology.

FIG. 13 shows a flow diagram of an example process of manufacturing a device for bodily fluid drainage and bioelectrical monitoring, in accordance with the present technology.

FIG. 14 shows a flow diagram of an example process of inserting a device for bodily fluid drainage and bioelectrical monitoring, in accordance with the present technology.

The technologies described herein will become more apparent to those skilled in the art from studying the Detailed Description in conjunction with the drawings. Embodiments or implementations describing aspects of the invention are illustrated by way of example, and the same references can indicate similar elements. While the drawings depict various implementations for the purpose of illustration, those skilled in the art will recognize that alternative implementations can be employed without departing from the principles of the present technologies. Accordingly, while specific implementations are shown in the drawings, the technology is amenable to various modifications.

### DETAILED DESCRIPTION

Intracranial electroencephalography (iEEG) with a depth electrode has been shown to provide valuable diagnostic information in the acute neurocritical care setting for neurologically altered or comatose patients. However, iEEG for neuromonitoring in the acute setting remains underutilized. A primary reason for this underutilization is the fact that traditional depth electrode insertion requires a separate procedure, including a separate scalp incision, skull perforation, and penetration into the brain. In most cases, the risk associated with these additional procedures is deemed to outweigh the potential benefit of iEEG, and the procedure is not performed.

Among the population of altered or comatose patients that stand to benefit from iEEG, many undergo insertion of an external ventricular drain (EVD) for real-time monitoring of intracranial pressure (ICP) and drainage of cerebrospinal fluid (CSF) for ICP relief. In this procedure, a twist-drill hole is made in the skull and an approximately 2.5-3.5 mm diameter silicone tube is passed through the cortex into the cerebral ventricle. Insertion of EVDs is common; it is estimated that around 25,000-35,000 EVDs are placed annually in the United States, with the frequency rising over time. Neurosurgical practitioners are universally familiar with EVD insertion and management. It is a component of guideline recommendations for the management of patients with aneurysmal subarachnoid hemorrhage and for the management of severe traumatic brain injury (TBI).

The utility of continuous scalp EEG monitoring in comatose patients with high likelihood of occult seizure activity is widely accepted among neurocritical care centers. However, there is emerging evidence that intracranial EEG provides more information than scalp EEG in many cases. For example, one study compared scalp EEG with intracranial EEG in 14 patients with brain injury and found that intracranial EEG detected seizures in 8 of the 14 patients that were not detectable on scalp EEG. Another study of 48 patients with various degrees of coma associated with subarachnoid hemorrhage found that intracranial EEG monitoring was far more sensitive in detecting seizures than scalp EEG: 18/48 patients showed seizure activity from intracranial EEG, while only 4/48 showed seizure activity from scalp EEG. Of note, the majority of these patients would warrant EVD placement as a component of guideline recommendations for the management of subarachnoid hemorrhage. The detection and treatment of seizures in the acute brain injury setting have tangible effects on several clinical outcome measures. However, iEEG remains underutilized, and procedural morbidity associated with its insertion remains a key barrier. The technology disclosed herein addresses this problem by combining iEEG with EVD insertion in a single procedure with no additional procedural morbidity.

The present patent document discloses technology that integrates two critical neurocritical care functionalities—ICP monitoring and CSF diversion—with iEEG monitoring into a single device. The device includes a dual-function elastomer tube that combines ventricular access for ICP monitoring and CSF diversion (the functions of a traditional EVD) with embedded circuitry enabling iEEG (the function of a depth electrode) that is implanted in a single procedure through a single scalp incision, skull perforation, and cortical penetration. The device is configured to minimize or eliminate additional procedural risk beyond that of a typical EVD insertion and to maximize compatibility with the typical surgical technique of implanting an EVD to ensure that the device is easily integrated into routine surgical practice without a significant learning curve. The disclosed embodiments, among other features and benefits, enable an improved safety profile for iEEG monitoring in critically neurologically injured patients and thereby encourage more widespread adoption of iEEG as a neuromonitoring modality. Furthermore, EVD catheters are frequently used in clinical scenarios other than for ventricular access, including as postoperative drains in the subdural space following subdural hematoma evacuation. There is a growing body of evidence that electrophysiologic derangements are common in these settings as well. Thus, an integrated catheter and iEEG system, as disclosed herein, has the potential for use even outside of standard EVD insertions for ventricular access.

Advantages of the disclosed technology include, among other features and benefits, enabling seamless neuromonitoring and therapeutic intervention for altered or comatose patients while eliminating the need for a separate procedure for insertion of an iEEG electrode. Additional advantages of the disclosed technology include a device that: (i) functions as a traditional EVD to monitor and relieve elevated intracranial pressure by draining CSF; (ii) provides iEEG functionality via embedded flexible microcircuitry and recording electrodes positioned along the catheter, allowing real-time monitoring of cortical and subcortical brain activity; (iii) minimizes additional procedural risk by leveraging the existing workflow of EVD insertion, requiring only a single scalp incision, twist-drill skull perforation, and cortical penetration; and (iv) simplifies implantation with a specialized trocar capable of simultaneously tunneling the drainage tube and the iEEG connector, ensuring efficient and reliable placement. By addressing the underutilization of iEEG monitoring in critical care settings, the disclosed technology combines diagnostic and therapeutic functions into a single streamlined device, reducing surgical complexity, improving safety, and facilitating broader adoption of iEEG neuromonitoring. Furthermore, the disclosed technology offers a novel integration of EVD and iEEG functionalities in a single device. By combining these functions, the device eliminates the need for a separate iEEG implantation procedure, which traditionally requires an additional scalp incision, skull perforation, and cortical penetration. This approach reduces patient risk, minimizes procedural complexity, and facilitates broader adoption of iEEG in critical care settings.

In an example embodiment of a device based on the disclosed technology, the device includes a flexible catheter that is inserted into the brain to allow doctors to perform two critical functions simultaneously: drain excess fluid from the brain and monitor brain activity. Typically, these functions require two separate devices and two different procedures, which can be risky and complicated. The device combines both functions into a single tube that can be inserted in one procedure, making it safer and simpler for surgeons and patients alike. The catheter includes small openings that drain excess fluid from the brain, helping to relieve pressure, and also includes tiny sensors (e.g., recording electrodes) along its surface that monitor electrical signals from the brain. This combined functionality allows physicians to better monitor and treat a patient's brain function without the need for multiple surgeries. Such a device makes brain monitoring and treatment more straightforward, safer, and more effective for critically ill patients.

### Dual-Use Ventricular Drain and Intracranial Electroencephalography Device

In the description that follows, example implementations of devices based on the disclosed technology are provided. In an embodiment, during an EVD procedure, a twist-drill hole is made in the patient's skull, and the elastomer tube device is inserted into the lateral ventricle using standard surgical techniques. Once inserted, the tube allows for CSF drainage through the perforations, which flows through the lumen to be measured and regulated for intracranial pressure management. The embedded recording electrodes, positioned anywhere along the shaft (e.g., near the cortical surface), allow for continuous electrophysiological monitoring of neural activity, providing valuable real-time insights into the patient's neurological status. The proximal end of the elastomer tube, along with the linear iEEG connector, is tunneled (e.g., navigated) under and/or through the scalp using the trocar, creating an easy-to-access port for both CSF drainage and iEEG data acquisition.

In the existing art, standard EVD catheters used in neurocritical care and neurosurgical settings for CSF drainage and ICP monitoring lack any capability for electrophysiological recording. Additionally, existing iEEG devices, such as depth electrodes, require separate procedures for placement and are typically used in controlled epilepsy monitoring units rather than acute critical care settings. The need for additional incisions and the complexity of separate electrode placement present significant barriers to their use in acute neurocritical care settings. The disclosed technology can be embodied in implementations to address these challenges. For example, in some devices based on the disclosed technology, the device includes a single lumen, and recording contacts are printed on a flexible and thin elastomer base such that the overall diameter of the device is approximately the same as standard EVDs and thus no additional procedural risk is posed. The recording contacts can be embedded directly onto the outer surface of the elastomer catheter, eliminating the need for a secondary lumen and ensuring direct contact between the recording electrodes and brain tissue. This design improves both safety and signal fidelity. As another example, some disclosed devices may include a trocar to facilitate tunneling of the entire device. Embodiments of the disclosed technology can also provide continuous iEEG capabilities without the need for a separately insertable or removable component.

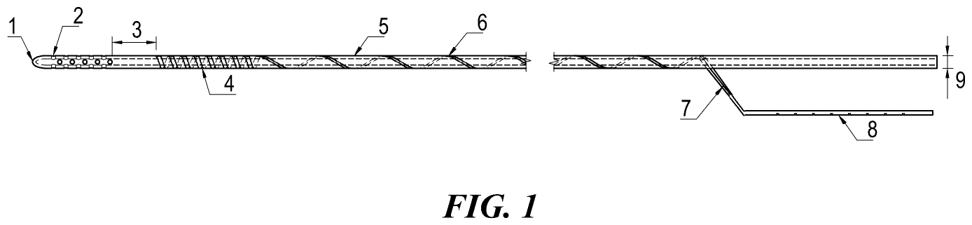

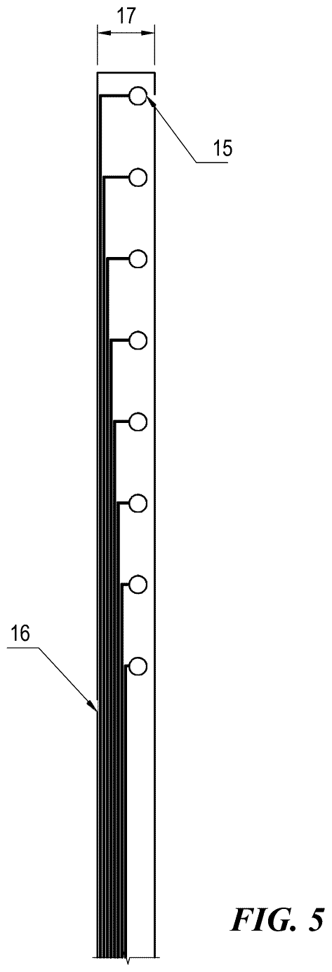

FIG. 1 shows a schematic of an example device, according to some embodiments of the present technology. In this example, the device includes an elastomer tube (5) approximately 300 mm in total length, about 3 mm in outer diameter (9), and about 1.5 mm in inner diameter. The distal portion of the flexible tube includes a distal tip (1) (e.g., at the distal end of the flexible tube) having a smooth conical shape that facilitates insertion of the device with minimal trauma to the brain using a rigid inner cannula (not shown) that is removed once the tip is at the intended depth. The distal portion of the catheter is inserted into the lateral ventricle of the brain via a twist-drill hole made in the skull using standard techniques. The distal portion of the tube contains a series of circular perforations (2) that allow CSF to flow into and up the tube to the proximal end, where the pressure can be transduced (e.g., with a manometer or electrical pressure transducer) and CSF can be drained as needed. At any desired distance (3) from the distal tip (e.g., approximately 40 mm from the distal tip), a series of metallic contacts (4) is arranged along the outside of the tube that serve as recording electrodes. For example, and as illustrated in FIG. 5, the metallic contacts (4) can include circular contacts arranged along the outside of the flexible tube. These contacts are connected via embedded microelectronics (6) (e.g., circuitry) that extend up the shaft of the tube toward the proximal end until the microelectronics branch off (7) to a linear connector (8) (e.g., an electrical connector) that can be plugged into an external signal processing unit via connection pins. The linear connector (8) can then be interfaced with external signal processing equipment for electrophysiological data collection and analysis. The metallic contacts (4) serve as iEEG recording electrodes, enabling real-time monitoring of brain activity. In some example embodiments, the metallic contacts are flexible, which allows them to conform to the shape of the elastomer tube, ensuring good electrical contact while minimizing irritation and mechanical stress on the surrounding brain tissue.

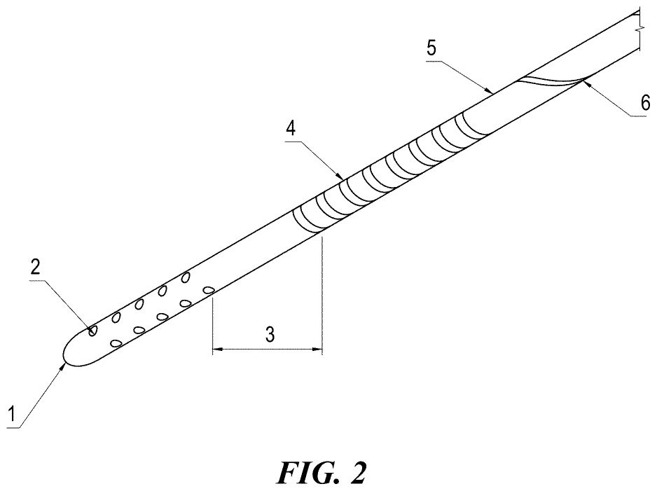

FIG. 2 shows another schematic view of the example device previously described with respect to FIG. 1, according to some embodiments of the present technology. FIG. 2 shows a conical tip disposed at a distal end of a distal portion of the example device, a plurality of perforations (2) at the distal portion of the example device, one or more metallic contacts (4) disposed on the example device at some distance (3) above the distal tip, and microelectronics (6) disposed along the example device.

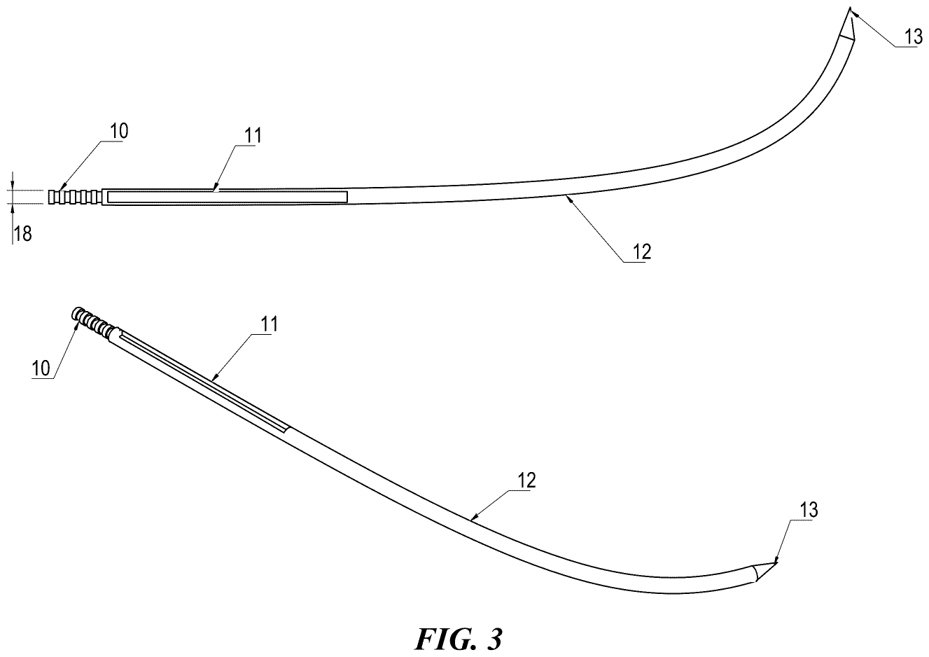

FIG. 3 shows a schematic of another example embodiment that can be implemented with the device of FIG. 1, according to some embodiments of the present technology. Consistent with standard EVD insertion technique, once the distal portion of the tube is inserted into the brain, the proximal end is tunneled underneath the scalp and exits the skin through a separate stab incision with the use of a trocar. The device may include a trocar, as shown in FIG. 3, capable of tunneling both the proximal tube end and the linear connector (8) in a single pass, simplifying the procedure and reducing the need for additional surgical steps. The metallic trocar (12) is approximately 150 mm long with an asymmetric curve. The distal end of the trocar includes a barbed connector (10) over which the proximal end of the elastomer drainage tube is fitted and pulled during the tunneling process. The barbed connector (10) can have an outer diameter (18) that is slightly larger than the inner diameter of the flexible tube (5) to facilitate a secure connection between the trocar (12) and the flexible tube (5). For example, the inner diameter of the flexible tube (5) can be approximately 1.5 mm, and the outer diameter (18) of the barbed connector (10) can be approximately 2.25 mm (e.g., between 2 mm and 2.5 mm). The outer diameter (18) can be approximately 150% of the size of the inner diameter of the flexible tube (5) (e.g., 130%-170% of the size of the inner diameter) and/or approximately 1.25 mm larger (e.g., between 0.5 mm and 1.5 mm larger) than the inner diameter of the flexible tube (5). Just proximal to the barbed connector is a recessed groove (11) into which the linear connector (8) fits, thus enabling both the proximal end of the drainage tube and the linear connector (8) to be pulled together during tunneling. The proximal end of the trocar is sharp (13) to facilitate skin perforation at the exit site.

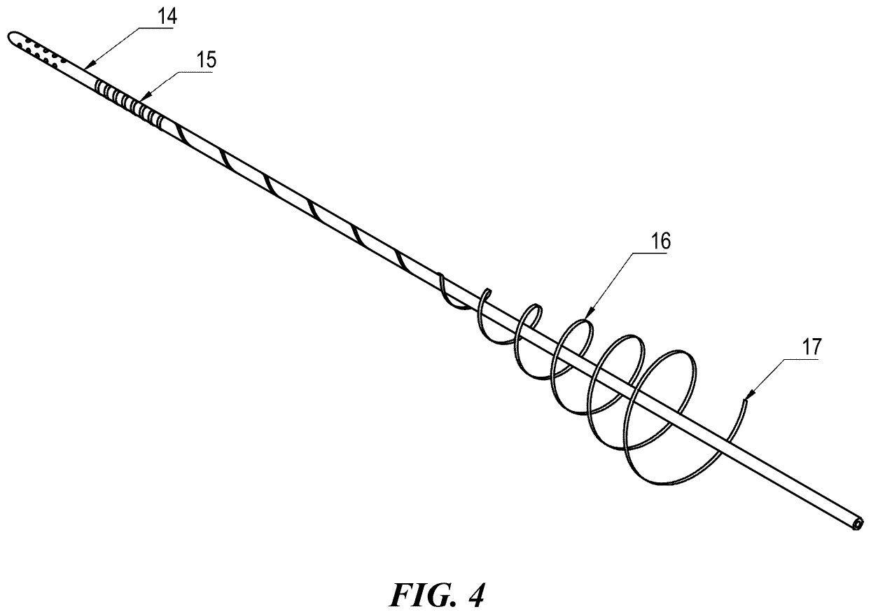

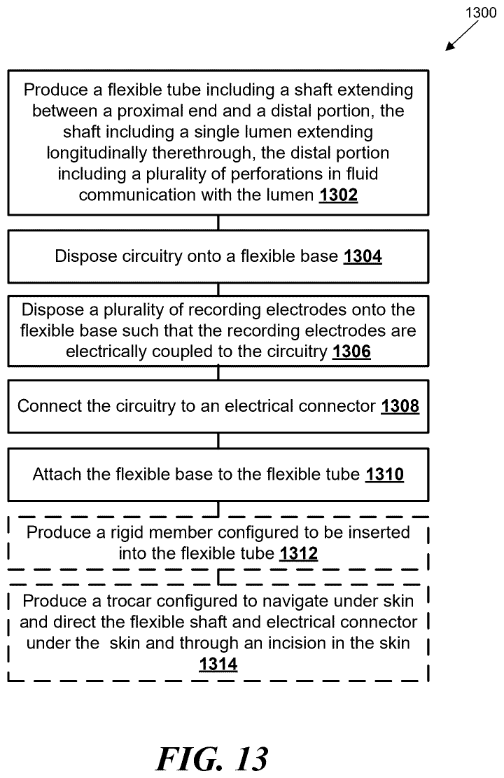

FIG. 4 shows a schematic of an elastomer tube containing distal perforations and bonded with a thin film substrate containing embedded recording contacts and associated circuitry, according to some embodiments of the present technology. The microelectronics (16) run up the shaft of the tube (14) as further shown in FIG. 4. As described in more detail with respect to FIG. 13 below, such configurations can result from an example embodiment that relates to a manufacturing process that involves designing and printing flexible microcircuitry onto and/or into an elastomer base (17) that is then mounted to a cylindrical drain (e.g., the elastomer tube (5) described with respect to FIG. 1), according to some embodiments of the present technology. As shown in FIG. 4, the elastomer base (17) can be attached in a helical pattern around the tube (14).

FIG. 5 shows a portion of the example embodiment depicted in FIG. 4, according to some embodiments of the present technology. As shown in FIG. 5, the recording contacts (15) are made of flexible materials that are mounted or printed onto an elastomer base, and the connections (16) (e.g., circuitry) are also mounted in a bundle that travels up the shaft (14) before branching off (7) to attach to the connector (8). This process allows for flexible electrode configurations, including directional contacts, microcontacts, or other customizable components. The width (17) of the elastomer base can be chosen based on the particular configuration. For example, the width (17) of the elastomer base can be between 1 mm and 2 mm.

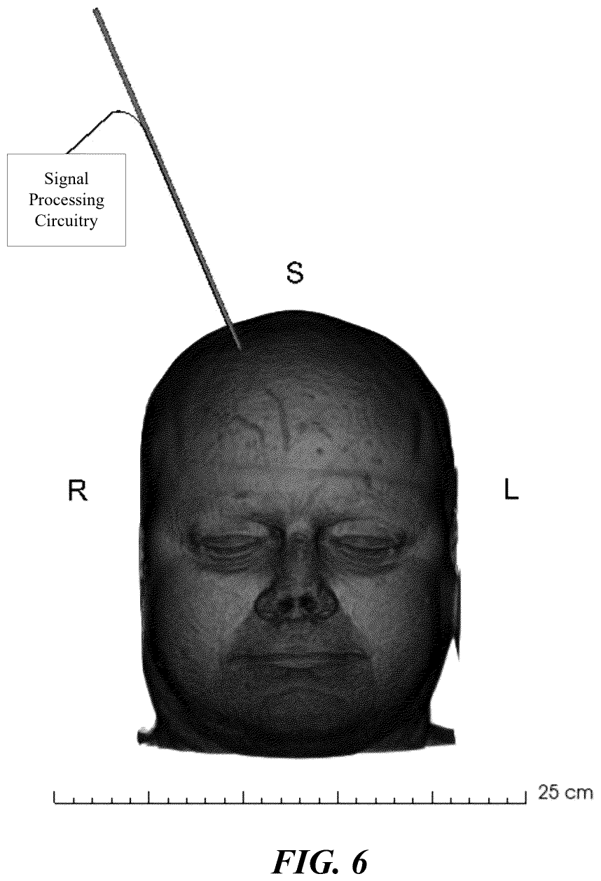

FIG. 6 shows an example insertion of a device according to embodiments of the present technology. An exemplary insertion location can be substantially similar and/or identical to that of a standard EVD. As illustrated, the device is operably connected to external signal processing circuitry. For instance, the signal processing circuitry is configured to process detected and/or received bioelectrical signals into intracranial electroencephalography (iEEG) signals.

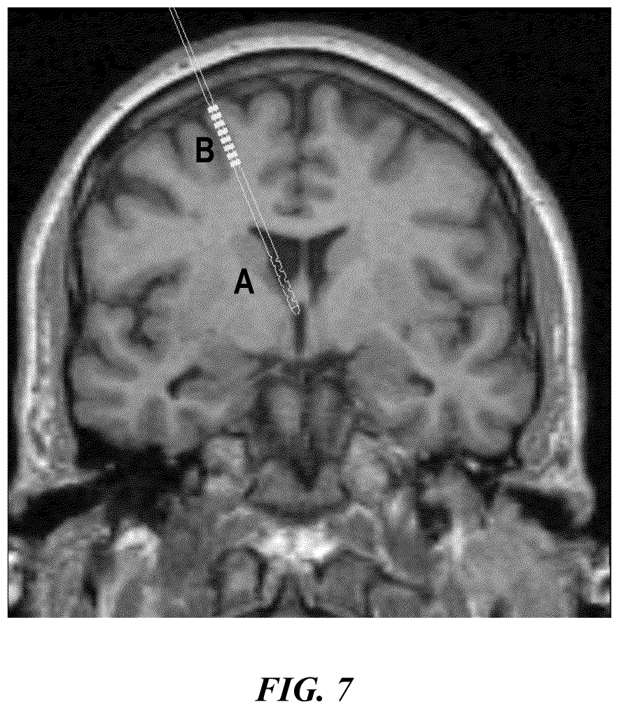

FIG. 7 shows an example of intracranial components of the device according to some embodiments of the present technology. As shown in FIG. 7, when the distal portion of the catheter has been inserted into the right lateral ventricle, distal perforations A enable CSF drainage through the tube, and a set of eight contacts B sits near the cortical surface to enable monitoring of electrophysiologic activity.

### Additional Embodiments

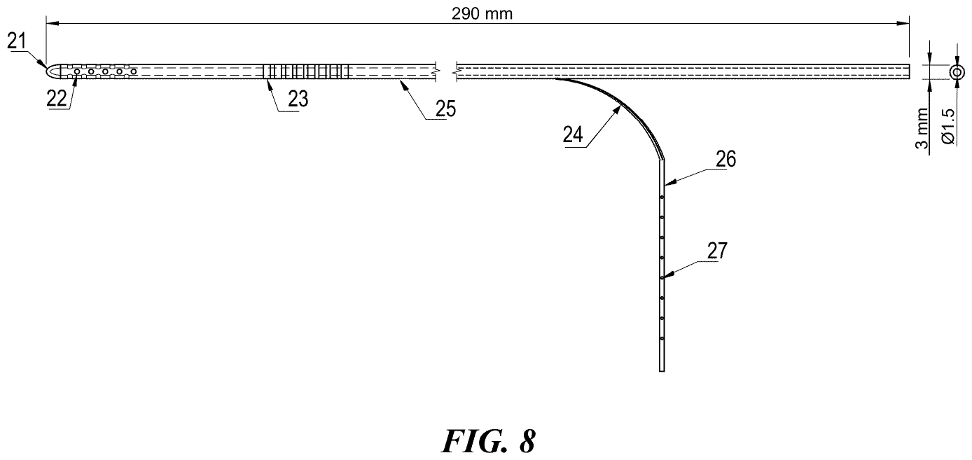

FIG. 8 shows a schematic of an example device, according to some embodiments of the present technology. In this example, the device includes a flexible tube (25) (e.g., an elastomer tube) of about 290 mm in total length, about 3 mm in outer diameter, and about 1.5 mm in inner diameter. The distal portion of the flexible tube includes a distal tip (21) (e.g., at the distal end of the flexible tube) having a smooth conical shape that can facilitate insertion of the device into an anatomical structure (e.g., the brain of a patient). Insertion of the distal portion can be performed using a rigid member (not shown) which is inserted into the flexible tube (25) during insertion of the distal portion and is removed once the tip is at the intended depth. The distal portion of the tube contains a series of perforations (22) that allow bodily fluid to flow into and up the tube to the proximal end, where the pressure can be transduced (e.g., with a manometer or electrical pressure transducer) and bodily fluid can be drained as needed. At a distance from the distal tip (e.g., about 40 mm), a series of recording electrodes (23) is arranged along the outside of the tube. These recording electrodes (23) are connected via circuitry (28) that extends up the shaft of the tube toward the proximal end until the circuitry branches off (24) to an electrical connector (26) (e.g., a linear connector) that can be plugged into an external signal processing unit via connection pins (27). The electrical connector (26) can then be interfaced with external signal processing equipment for electrophysiological data collection and analysis. The recording electrodes (23) serve as iEEG recording electrodes, enabling real-time monitoring of brain activity. In some example embodiments, the recording electrodes are flexible, which allows them to conform to the flexible tube (25), ensuring good electrical contact while minimizing irritation and mechanical stress. For example, the recording electrodes (23) can be rectangular and flexible such that the recording electrodes (23) wrap around the flexible tube (25).

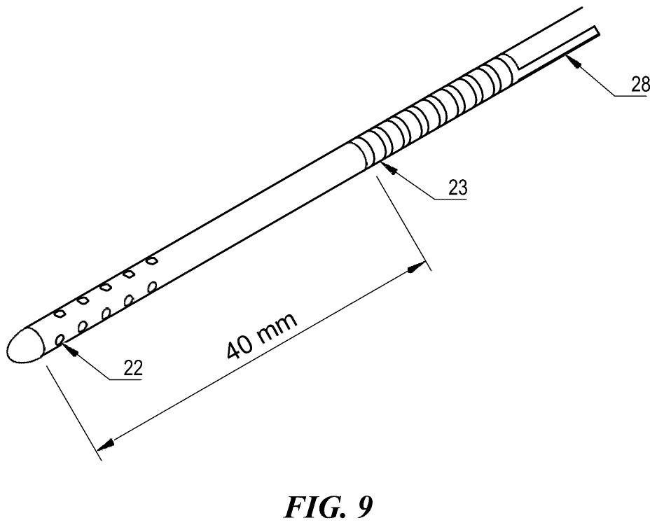

FIG. 9 shows another schematic view of the example device previously described with respect to FIG. 8, according to some embodiments of the present technology. FIG. 9 shows a conical tip disposed at a distal end of a distal portion of the example device, a plurality of perforations (22) at the distal portion of the example device, one or more recording electrodes (23) disposed on the example device, and circuitry (28) disposed along the example device.

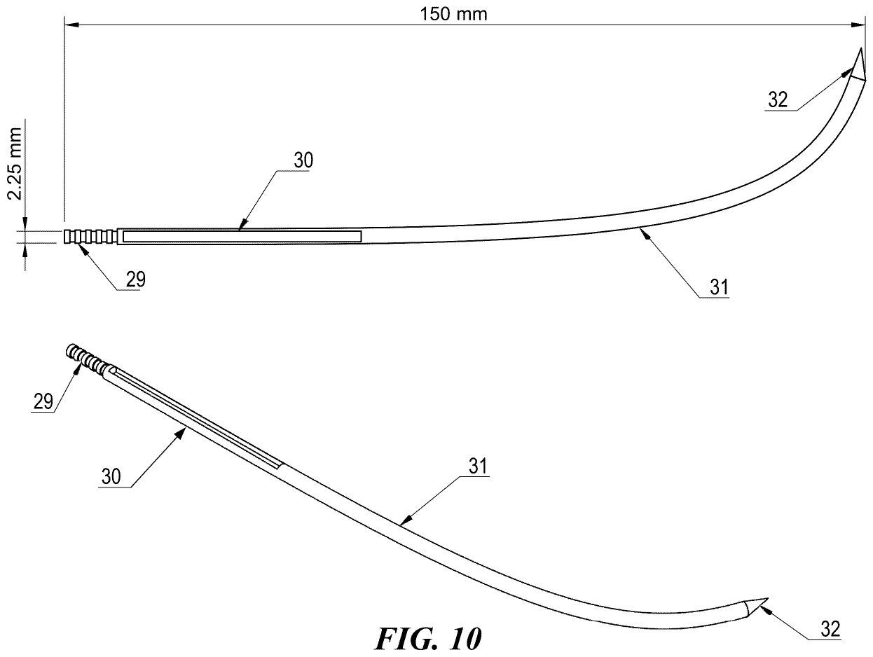

FIG. 10 shows a schematic of another example embodiment that can be implemented with the device of FIG. 8, according to some embodiments of the present technology. Once the distal portion of the flexible tube (25) is inserted into an anatomical structure, the proximal end can be tunneled underneath the skin of the patient and can exit the skin through a separate incision. This can, for example, reduce the risk of infection (e.g., of the anatomical structure) by preventing direct access to the anatomical structure from external sources. For example, consistent with standard EVD insertion technique, the distal portion of the tube can be inserted into the brain, and the proximal end can be tunneled underneath the scalp to exit the skin through a separate stab incision. The proximal end can be tunneled under the skin with the use of a trocar, such as the example trocar shown in FIG. 10, capable of tunneling both the proximal tube end and the electrical connector (26) in a single pass. The trocar (31) can be approximately 150 mm long and/or have an asymmetric curve. The distal end of the trocar can include a barbed connector (29) over which the proximal end of the flexible tube can be fitted and pulled during the tunneling process. Proximal to the barbed connector is a recessed groove (30) into which the electrical connector (26) fits, thus enabling both the proximal end of the flexible tube (25) and the electrical connector (26) to be pulled together during tunneling. The proximal end of the trocar is sharp (32) to facilitate skin perforation at the exit site.

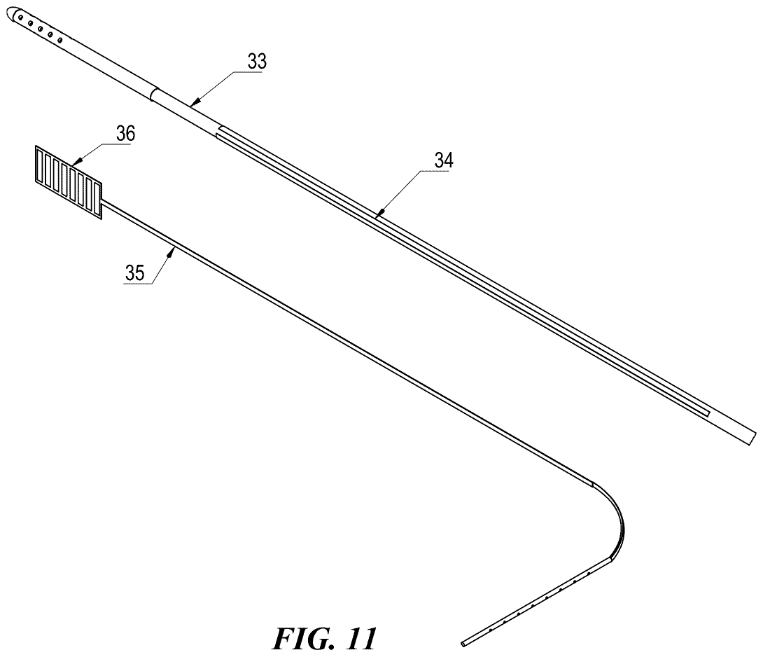

FIG. 11 shows a schematic of a flexible tube, with distal perforations, that is produced with a slight relief at the site of recording electrodes (33), according to some embodiments of the present technology. The circuitry runs up the shaft of the flexible tube (34) as further shown in FIG. 11. As described in more detail with respect to FIG. 13 below, such configurations can result from an example embodiment which relates to a manufacturing process that involves designing and printing flexible microcircuitry onto and/or into a flexible base (e.g., an elastomer base) that is then mounted to a cylindrical drain (e.g., the flexible tube (25) described with respect to FIG. 8), according to some embodiments of the present technology.

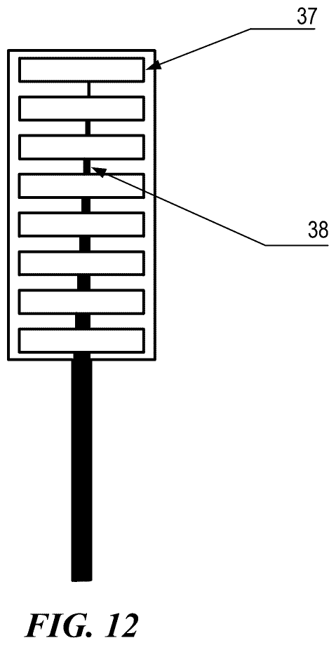

FIG. 12 shows a portion of the example embodiment depicted in FIG. 11, according to some embodiments of the present technology. As shown in FIG. 12, the recording electrodes (37) are made of flexible materials that are mounted or printed onto a flexible base (36), and connections (38) (e.g., circuitry) are also mounted in a bundle that travels up the shaft (35) before branching off (24) to attach to the electrical connector (26). This process allows for flexible electrode configurations, including directional contacts, microcontacts, or other customizable components.

### Manufacturing Method Flow Diagram

FIG. 13 shows a flow diagram of an example process 1300 of manufacturing a device for bodily fluid drainage and bioelectrical monitoring, in accordance with the present technology. The device can be used for bodily fluid drainage, bodily fluid monitoring (e.g., fluid pressure monitoring), and/or bioelectrical monitoring in various anatomical structures. In some embodiments, the device is inserted into a fluid-containing anatomical structure of a subject to monitor bodily fluid pressure and/or drain bodily fluid from the anatomical structure while simultaneously recording bioelectrical signals from tissue adjacent to and/or within the anatomical structure. For example, the device can be inserted into a ventricle of the brain to drain cerebrospinal fluid while recording intracranial electroencephalography (iEEG) signals from cortical and/or subcortical tissue. Alternatively, or additionally, the device can be inserted into a subdural space following hematoma evacuation to drain fluid while monitoring electrophysiologic activity. The device can use and/or include one or more components described with respect to FIGS. 1-7, including the elastomer tube (5), metallic contacts (4), and/or trocar (12), and/or components described with respect to FIGS. 10-12, including the flexible tube (25), recording electrodes (23), and/or trocar (31).

The process 1300 includes, at 1302, producing a flexible tube (e.g., the elastomer tube (5) described with respect to FIG. 1 and/or the flexible tube (25) described with respect to FIG. 10). The flexible tube can include a shaft extending between a proximal end and a distal portion. The shaft can include a single lumen extending longitudinally along and/or through the shaft and providing fluid connection between the distal portion and the proximal end. The distal portion can be configured to be inserted into a fluid-containing anatomical structure of a subject. The distal portion can include a plurality of perforations in fluid communication with the lumen (e.g., enabling bodily fluid to flow from the anatomical structure through the perforations into the lumen and travel through the lumen to the proximal end). In some embodiments, the distal portion of the flexible tube includes a distal tip (e.g., at a distal end of the distal portion). The distal tip can be configured in a conical shape (e.g., a rounded conical shape) to facilitate insertion with minimal trauma. In some embodiments, the flexible tube may have dimensions of about 300 mm in total length, about 3 mm in outer diameter (e.g., less than 3.5 mm, such as between 2.5 mm and 3.5 mm, and/or less than 3 mm), and about 1.5 mm in inner diameter (e.g., between 1 mm and 2 mm). For example, the lumen can have a diameter that is between 1 mm and 2 mm. In some embodiments, the flexible tube can be made of an elastomer material. In some embodiments, the flexible tube can have a relief and/or indentation on the outer surface of the shaft that is configured to receive electrical circuitry (e.g., a flexible base with recording electrodes and/or circuitry disposed thereon).

The process 1300 includes, at 1304, disposing circuitry (e.g., microelectronic circuitry) onto a flexible base (e.g., the elastomer base (17) described with respect to FIG. 4 and/or the flexible base (36) described with respect to FIG. 12). For example, the circuitry can be mounted and/or printed onto the flexible base. The flexible base can be configured to be affixed onto the outside of the shaft of the flexible tube (e.g., such that a portion of the flexible base wraps around the shaft). The circuitry is configured to convey bioelectrical signals and can be flexible. In some embodiments, the flexible base comprises an elastomer material.

The process 1300 includes, at 1306, disposing a plurality of recording electrodes onto the flexible base. For example, the recording electrodes can be mounted and/or printed onto the flexible base. The recording electrodes can be configured to record bioelectrical signals. For example, the recording electrodes can be configured to record bioelectrical signals of tissue adjacent to or within an anatomical structure, such as tissue contacting and/or in electrical communication with the recording electrodes. The plurality of recording electrodes is electrically coupled to the circuitry and can be configured to record bioelectrical signals by conveying bioelectrical signals from tissue to the circuitry. In some embodiments, the plurality of recording electrodes includes metallic contacts (e.g., flexible metallic contacts and/or metallic contacts (4) described with respect to FIG. 1). In some embodiments, the recording electrodes can include directional contacts and/or microcontacts.

The process 1300 includes, at 1308, connecting the circuitry to an electrical connector. The electrical connector can include at least one conductive element (e.g., one or more electrical connection pins). The electrical connector is configured to interface with signal processing circuitry for bioelectrical data collection and analysis. For example, the electrical connector can be configured to interface with and/or couple to a device used to process the bioelectrical signals into EEG signals (e.g., iEEG signals) and record, store, and/or display such EEG signals.

The process 1300 includes, at 1310, attaching the flexible base to the flexible tube. For example, a portion of the flexible base (e.g., a portion of the flexible base with the recording electrodes disposed thereon and/or a portion of the flexible base with a portion of the circuitry disposed thereon) can be attached to the flexible tube (e.g., attached onto an outside surface of the flexible tube and/or inserted into a relief in the flexible tube). In some embodiments, attaching the flexible base can include adhesive bonding, thermal bonding, overmolding, and/or chemical bonding. In some embodiments, the flexible base is attached in a helical pattern, such that the flexible base (e.g., a portion of the flexible base with the plurality of recording electrodes disposed thereon and/or a portion of the flexible base with a portion of the circuitry disposed thereon) extends along the flexible tube while wrapping around the flexible tube.

In some embodiments, a portion of the circuitry (e.g., a portion of the flexible base with a portion of the circuitry disposed thereon) is not attached to the flexible tube and branches (e.g., extends from) from the flexible tube (e.g., at a location on the flexible tube that is between the proximal end and the recording electrodes). For example, the flexible base can be attached to the flexible tube such that a first portion of the circuitry (e.g., disposed on a first portion of the flexible base) is disposed on the shaft of the flexible tube while a second portion of the circuitry (e.g., disposed on a second portion of the flexible base) branches from the shaft at a location that is between the plurality of the recording electrodes and the proximal end of the flexible tube. The circuitry can branch from the flexible tube and connect to and/or electrically couple to the electrical connector (e.g., such that the electrical connector is attached to the flexible tube via the branching circuitry and/or branching flexible base but otherwise can freely move independently of the flexible tube). For example, the electrical connector can include at least one conductive element (e.g., a connector pin) electrically connected to the circuitry (e.g., to the portion of the circuitry extending from the flexible tube). In some embodiments, connecting the circuitry to the electrical connector at 1308 may occur after attaching the flexible base to the flexible tube at 1310.

While the example process 1300 describes disposing recording electrodes and/or circuitry onto the flexible tube by disposing the recording electrodes and/or circuitry onto the flexible base, the present technology is not so limited. For example, a portion of the recording electrodes and/or a portion of the circuitry can be disposed (e.g., via mounting and/or printing) directly onto the flexible tube (e.g., an outer surface of the flexible tube). In some implementations, a portion of the recording electrodes and/or a portion of the circuitry is at least partially disposed directly onto the flexible tube and/or at least partially disposed onto the flexible base (e.g., branching and/or extending from the flexible tube).

In some embodiments, the process 1300 includes, at 1312, producing a rigid member configured to be inserted into the flexible tube (e.g., inserted into the lumen, inserted through the proximal end of the flexible tube, and/or inserted into the distal portion of the flexible tube). The rigid member can be configured to facilitate the insertion of the flexible tube into the anatomical structure by providing rigidity to the flexible tube during insertion. In some embodiments, the rigid member is a cannula configured to allow bodily fluid to flow through the rigid member and/or the lumen during and/or after insertion of the flexible tube into the anatomical structure.

In some embodiments, the process 1300 includes, at 1314, producing a trocar (e.g., trocar (12) described with respect to FIG. 3 and/or trocar (31) described with respect to FIG. 10). The trocar can be configured to navigate under the skin of the patient and/or direct the flexible shaft (e.g., the proximal end of the flexible shaft) and/or the electrical connector under the skin and/or through an incision in the skin. The trocar can include a shaft (e.g., a curved shaft) extending between a proximal end and a distal portion. In some embodiments, the shaft includes an asymmetric curve (e.g., with curvature increasing towards the proximal end). In some embodiments, the trocar is produced with a proximal end configured to perforate skin (e.g., a sharp proximal end). In some embodiments, a distal portion includes a mechanical connector (e.g., a barbed connector) at the distal end, configured to engage (e.g., attach and/or mechanically couple to) the proximal end of the flexible tube (e.g., have the proximal end of the flexible tube pulled over the mechanical connector such that the mechanical connector is at least partially disposed within the lumen of the flexible tube). In some embodiments, the trocar has a recessed groove configured to receive at least one of the circuitry (e.g., a portion of the circuitry) and/or the electrical connector, or a portion thereof. For example, the recessed groove can be configured to receive the electrical connector (e.g., a portion of the electrical connector) and/or the electrical connector and a portion of the circuitry. The recessed groove can be configured such that the circuitry and/or the electrical connector remains fixed in the groove while the trocar is navigated under the skin and/or through the incision in the skin. In some embodiments, the trocar has dimensions of approximately 150 mm in length and includes a shaft with a diameter of approximately 3 mm (e.g., a diameter matching the diameter of the flexible tube).

It is contemplated that the operations and/or descriptions of FIG. 13 may be used with any other embodiment of this disclosure. In addition, the operations and descriptions described in relation to FIG. 13 may be done in alternative orders, in parallel, or multiple times to further the purposes of this disclosure. For example, each of these operations may be performed in any order, in parallel, or simultaneously to reduce delay or increase the speed of the process. Furthermore, it should be noted that any of the devices or equipment discussed in relation to the other figures or otherwise disclosed herein could be used to perform one or more of the operations in FIG. 13.

### Insertion Method Flow Diagram

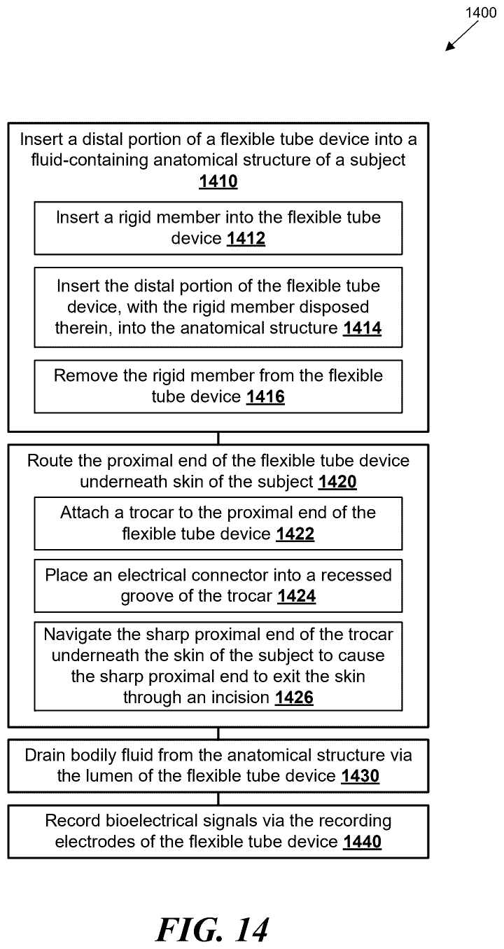

FIG. 14 shows a flow diagram of an example process 1400 of inserting a device for bodily fluid drainage and bioelectrical monitoring, in accordance with the present technology. The process 1400 can be performed using devices and/or components described with respect to FIGS. 1-12 and/or manufactured according to the process 1300 described with respect to FIG. 13.

The process 1400 includes, at 1410, inserting a distal portion of a flexible tube device into a fluid-containing anatomical structure of a subject. The flexible tube device includes a proximal end, a shaft extending between the proximal end and the distal portion, a lumen extending longitudinally through the shaft, a plurality of perforations in fluid communication with the lumen and disposed at the distal portion, and recording electrodes disposed along the shaft. In some embodiments, the flexible tube device includes a conical tip (e.g., at a distal end of the distal portion) configured to facilitate insertion into the anatomical structure. In some embodiments, the flexible tube device includes electrical circuitry (e.g., disposed on an outer surface of the shaft) electrically coupled to the recording electrodes and extending along a portion of the shaft, such that the electrical circuitry branches from the shaft at a location on the shaft that is between the proximal end and the recording electrodes and is electrically and/or mechanically coupled to an electrical connector (e.g., linear connector (8) as described in more detail with respect to FIG. 1 and/or electrical connector (26) as described with respect to FIG. 10).

In some embodiments, the anatomical structure can include a lateral ventricle of a brain (e.g., the anatomical structure being the brain). For example, inserting the distal portion of the flexible tube device into the anatomical structure can include making a twist-drill hole in the skull of the subject and inserting the distal portion of the flexible tube device through the twist-drill hole into the lateral ventricle of the brain. Inserting the distal portion of the flexible tube device into the brain can be performed using standard surgical techniques (e.g., techniques used to insert external ventricular drains (EVDs)).

As an illustrative example of inserting the distal portion of the flexible tube device into the fluid-containing anatomical structure, the process 1400 can include, at 1412, inserting a rigid member into the lumen of the flexible tube device via the proximal end. The rigid member can facilitate insertion of the distal portion into the anatomical structure of the subject (e.g., by providing rigidity to the flexible tube device during insertion). In some embodiments, the rigid member is a cannula (e.g., configured to allow fluid to flow therethrough). The process 1400 can include, at 1414, inserting the distal portion of the flexible tube device, with the rigid member disposed therein, into the anatomical structure. The process 1400 can include, at 1416, removing the rigid member from the flexible tube device subsequent to inserting the distal portion into the anatomical structure (e.g., such that the flexible tube device remains inserted into the anatomical structure).

In some embodiments, the process 1400 includes, at 1420, routing the proximal end of the flexible tube device underneath skin of the subject. The proximal end of the flexible tube can be routed under the skin from an original location (e.g., where the distal portion of the flexible tube device is inserted into the anatomical structure) to an exit location (e.g., a perforation and/or stab incision in the skin). Routing the proximal end underneath the skin can, for example, help reduce infection risk (e.g., by providing a smaller opening at the exit site and/or preventing direct exposure of the fluid-containing anatomical structure). In some embodiments, the flexible tube device is inserted into a lateral ventricle of a brain, and the proximal end of the flexible tube device is routed under the scalp of the subject and through a perforation in the scalp. In some embodiments, routing the proximal end underneath the skin is performed using a trocar (e.g., trocar (12) as described with respect to FIG. 3 and/or trocar (31) as described with respect to FIG. 12). The trocar can include a mechanical connector (e.g., a barbed connector) disposed at a distal portion (e.g., a distal end) of the trocar and/or a sharp proximal end configured to perforate skin.

As an illustrative example of routing the proximal end of the flexible tube device underneath skin of the subject, the process 1400 can include, at 1422, attaching a trocar to the proximal end of the flexible tube device. Attaching the trocar to the flexible tube device can include attaching the proximal end of the flexible tube device to a mechanical connector disposed at the distal end of the trocar (e.g., placing the shaft of the flexible tube device around the mechanical connector such that the mechanical connector is at least partially disposed within the lumen of the flexible tube device). The process 1400 can include, at 1424, placing at least a portion of an electrical connector branching from the shaft (e.g., as described with respect to 1410) and/or a portion of circuitry branching from the shaft into a recessed groove of the trocar. For example, the trocar can include a recessed groove configured to receive and/or secure the electrical connector and/or circuitry to the trocar. The process 1400 can include, at 1426, navigating the sharp proximal end of the trocar underneath skin of the subject to cause the sharp proximal end to exit the skin through an incision (e.g., an incision created by the sharp proximal end of the trocar). This can cause the proximal end of the flexible tube device and/or the electrical connector to extend under and/or be routed under the skin and through the incision (e.g., such that the electrical connector, proximal end of the flexible tube device, and/or the lumen of the flexible tube device are accessible through the incision in the skin).

The process 1400 includes, at 1430, draining bodily fluid from the anatomical structure via the flexible tube device (e.g., draining bodily fluid through the plurality of perforations into the lumen and towards the proximal end of the flexible tube device, where the bodily fluid exits the lumen through the proximal end of the flexible tube device). The bodily fluid can be collected in a collecting device (e.g., drainage bag, graduated collection chamber). For example, the process 1400 can include connecting (e.g., via one or more flexible connectors) a collection device to the proximal end of the flexible tube device and draining bodily fluid from the anatomical structure into the collection device. In some embodiments, the flexible tube device (e.g., the proximal end of the flexible tube device) and/or the collection device are connected to one or more pressure monitoring devices (e.g., a manometer and/or pressure transducer).

The process 1400 includes, at 1440, recording bioelectrical signals via the flexible tube device (e.g., recording bioelectrical signals using the recording electrodes, which are transmitted through the circuitry and/or the electrical connector into a signal processing device). For example, the process 1400 can include connecting (e.g., via the electrical connector) a signal processing device to the flexible tube device and processing, storing, and/or displaying signals. In some embodiments, the bioelectrical signals are processed (e.g., by a signal processing device) into EEG and/or iEEG signals.

It is contemplated that the operations and/or descriptions of FIG. 14 may be used with any other embodiment of this disclosure. In addition, the operations and descriptions described in relation to FIG. 14 may be done in alternative orders, in parallel, or multiple times to further the purposes of this disclosure. For example, each of these operations may be performed in any order, in parallel, or simultaneously to reduce delay or increase the speed of the process. Furthermore, it should be noted that any of the devices or equipment discussed in relation to the other figures or otherwise disclosed herein could be used to perform one or more of the operations in FIG. 14.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer-readable medium for execution by, or to control the operation of, data processing apparatus. The computer-readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including, by way of example, a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special-purpose logic circuitry, e.g., an FPGA (field programmable gate array) or an ASIC (application-specific integrated circuit).

Processors suitable for the execution of a computer program include, by way of example, both general and special-purpose microprocessors, and any one or more processors of any kind of digital computer. Generally, a processor will receive instructions and data from a read-only memory or a random-access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Computer-readable media suitable for storing computer program instructions and data include all forms of nonvolatile memory, media and memory devices, including, by way of example, semiconductor memory devices, e.g., EPROM, EEPROM, and flash memory devices. The processor and the memory can be supplemented by, or incorporated in, special-purpose logic circuitry.

Only a few implementations and examples are described, and other implementations, enhancements, and variations can be made based on what is described and illustrated in this patent document.

What is claimed is:

1. A device for bodily fluid drainage and bioelectrical monitoring, comprising:
a flexible tube having a shaft extending between a proximal end and a distal portion and a lumen extending longitudinally, the flexible tube configured for insertion into a fluid-containing anatomical structure of a subject, wherein: the distal portion of the flexible tube comprises a plurality of perforations in fluid communication with the lumen, the plurality of perforations configured to enable bodily fluid to flow from the anatomical structure into the lumen of the flexible tube and towards the proximal end of the flexible tube;
a plurality of recording electrodes disposed along an outer surface of the shaft and configured to record bioelectrical signals of tissue adjacent to or within the anatomical structure;
circuitry electrically coupled to the plurality of recording electrode, wherein a first portion of the circuitry is disposed along the shaft and a second portion of the circuitry branches from the shaft at a location that is between the plurality of recording electrodes and the proximal end of the flexible tube; and
an electrical connector configured to interface with signal processing circuitry, the electrical connector comprising at least one conductive element electrically connected to the second portion of the circuitry.

2. The device of claim 1, wherein the plurality of recording electrodes comprises metallic contacts configured to record bioelectrical signals of tissue in contact with or in electrical communication with the metallic contacts.

3. The device of claim 1, wherein the plurality of recording electrodes are flexible.

4. The device of claim 1, wherein a distal end of the flexible tube is of conical shape.

5. The device of claim 1 comprising a trocar having a distal end and a proximal end, wherein:
the trocar comprises a mechanical connector disposed at the distal end of the trocar, and
the proximal end of the trocar is configured to perforate skin.

6. The device of claim 5, wherein the mechanical connector of the trocar is a barbed connector configured to engage the proximal end of the flexible tube.

7. The device of claim 5, wherein the trocar comprises a recessed groove disposed along the trocar and configured to receive at least one of the connector or the second portion of the circuitry, or a portion thereof.

8. The device of claim 1, wherein the flexible tube comprises an elastomer material.

9. The device of claim 1, wherein the bioelectrical signals are intracranial electroencephalography (iEEG) signals, wherein the signal processing circuitry is configured to process the iEEG signals.

10. The device of claim 1, wherein a diameter of the shaft is between 2.5 mm and 3.5 mm.

11. The device of claim 1, wherein the plurality of recording electrodes are configured such that when the distal portion of the flexible tube is inserted into a lateral ventricle of a brain, the plurality of recording electrodes are disposed near a cortical surface of the brain.

12. The device of claim 1, wherein the circuitry extends along the flexible tube in a helical pattern.

13. A method for bodily fluid drainage and bioelectrical monitoring, comprising:
inserting a distal portion of a flexible tube of a device into a fluid-containing anatomical structure of a subject,
wherein the device comprises:
the flexible tube having a shaft extending between a proximal end and the distal portion and a lumen extending longitudinally through the shaft,
a plurality of perforations in fluid communication with the lumen and disposed at the distal portion, and
recording electrodes disposed along the shaft;
draining bodily fluid from the anatomical structure through the plurality of perforations and into the lumen towards the proximal end of the flexible tube; and
obtaining, via the recording electrodes, bioelectrical signals from tissue adjacent to or within the anatomical structure.

14. The method of claim 13, comprising:
inserting, into the lumen via the proximal end of the flexible tube, a rigid member, wherein the rigid member facilitates the insertion of the distal portion of the flexible tube device into the anatomical structure of the subject; and
subsequent to inserting the distal portion of the flexible tube device into the anatomical structure of the subject, removing the rigid member from the flexible tube.

15. The method of claim 13, comprising:
attaching, to the proximal end of the flexible tube, a trocar having a distal end and a proximal end, wherein:
the trocar comprises a mechanical connector disposed at the distal end of the trocar,
the proximal end of the trocar is configured to perforate skin, and
the attaching the trocar to the proximal end of the flexible tube comprises attaching the proximal end of the flexible tube to the mechanical connector disposed at the distal end of the trocar;
navigating the proximal end of the trocar underneath skin of the subject to cause the proximal end of the trocar to exit the skin of the subject through an incision created by the trocar; and
causing the proximal end of the flexible tube to extend under the skin of the subject and through the incision created by the trocar.

16. The method of claim 15, wherein the device comprises an electrical connector branching from the shaft and configured to convey electrical signals from the recording electrodes to one or more conductive elements disposed on the electrical connector, wherein the trocar comprises a recessed groove configured to receive the electrical connector while the proximal end of the flexible tube is attached to the mechanical connector of the trocar, the method comprising:
prior to navigating the proximal end of the trocar underneath the skin, placing the electrical connector in the recessed groove of the trocar; and
causing the electrical connector to pass through the incision created by the trocar.

17. The method of claim 13, wherein inserting the distal portion of the flexible tube into the anatomical structure of the subject is performed according to a procedure for inserting an external ventricular drain (EVD) into the subject.

18. A system comprising:
a device for bodily fluid drainage and bioelectrical monitoring, the device comprising:
a flexible tube configured for insertion into a fluid-containing anatomical structure of a subject, the flexible tube comprising a shaft extending between a proximal end and a distal portion, a lumen disposed inside of the shaft and extending longitudinally therethrough, and a plurality of perforations at the distal portion in fluid communication with the lumen and configured to enable bodily fluid to flow from the anatomical structure into the lumen and towards the proximal end;
a plurality of recording electrodes disposed along the shaft and configured to record bioelectrical signals of tissue adjacent to or within the anatomical structure;
circuitry disposed along the shaft and electrically coupled to the plurality of recording electrodes, wherein a portion of the circuitry extends from the shaft at a location along the shaft that is between the plurality of recording electrodes and the proximal end of the flexible tube; and
an electrical connector comprising electrical pins configured to electrically couple the circuitry to a signal processing device.

19. The system of claim 18, comprising a collection device configured to collect fluid from the anatomical structure and measure pressure of the collected fluid.

20. The system of claim 18, comprising a signal processing device electrically coupled to the circuitry and configured to process the bioelectrical signals into intracranial electroencephalography (iEEG) signals.
